(19) United States
(12) Patent Application Publication
(10) Pub. No.: US 2023/0248512 A1
(43) Pub. Date: Aug. 10, 2023

(54) TISSUE-BASED REINFORCED HEART VALVES

(71) Applicant: EDWARDS LIFESCIENCES CORPORATION, Irvine, CA (US)

(72) Inventors: Tara J. Tod, Riverside, CA (US); Mark Van Nest, Rancho Santa Margarita, CA (US); Kiem Bao Nguyen, Westminster, CA (US); Liqiong Gui, Irvine, CA (US); Lien Huong Thi Hoang, San Juan Capistrano, CA (US); Son V. Nguyen, Irvine, CA (US); Hao Shang, Irvine, CA (US)

(21) Appl. No.: 18/301,176

(22) Filed: Apr. 14, 2023

Related U.S. Application Data
(60) Provisional application No. 63/093,019, filed on Oct. 16, 2020.

(51) Int. Cl. A61F 2/24

(52) U.S. Cl. CPC A61F 2/2412; A61F 2250/0098

(57) ABSTRACT

Devices and methods for reinforcing a tissue-based heart valve are provided. A reinforced tissue valve can provide structure and rigidity to withstand stresses that occur within the vasculature. In some instances, thickened tissue provides structure or rigidity. In some instances, a biocompatible filler is utilized within folded, rolled, or layered tissue. In some instances, a mesh is included with a reinforced tissue valve for further strength. In some instances, a mesh frame is utilized along the sidewall of a tissue-based heart valve. In some instances, a leaflet assembly is provided within a conduit having thickened tissue at the leaflet commissures.

### CROSS REFERENCE TO RELATED APPLICATIONS

This application is a continuation of International Patent Application No. PCT/US2021/054,443, filed Oct. 11, 2021, which claims the benefit of U.S. Patent Application No. 63/093,019, filed Oct. 16, 2020, the entire disclosures all of which are incorporated by reference for all purposes.

### TECHNICAL FIELD

The application is generally directed to tissue-based heart valves for heart valve replacement.

### BACKGROUND

Valvular stenosis and regurgitation are a few of number of complications that may necessitate a heart valve replacement. Traditional replacement valves are constructed from various biocompatible metals, polymers and animal pericardium tissue. Pericardium tissue can be derived from various animals, including bovine, porcine, and equine.

### SUMMARY OF THE DISCLOSURE

The disclosure provides description of several tissue-based valvular devices for implantation. Generally, reinforcement is provided for tissue-based valvular devices in order to withstand the pulsatile pressures in the vasculature, especially within the aorta where pulsatile pressures are very high. Reinforcement of tissue-based valvular devices prevents and/or mitigates the valve from collapsing and helps maintain shape.

In an example of an implantable device for heart valve replacement, a prosthetic comprises a sidewall, a set of leaflets, and set of commissures adjoining the set of leaflets. The sidewall and each leaflet of the set of leaflets are composed of animal tissue. A reinforcing tissue structure is secured to the sidewall at the base of a set of leaflets. The reinforcing tissue structure comprises animal tissue having a thickness greater than the thickness of a thickness of the sidewall or the leaflets.

In an example of a reinforcing tissue for use with a prosthetic valve, an animal tissue has a length, a width, and a thickness. The tissue is contoured along its length such that the contour matches a plurality of cusp edges of a plurality of leaflets and a plurality of commissures of the plurality of leaflets of a heart valve.

In an example of an implantable heart valve device, a prosthetic heart valve comprises a sidewall, a set of leaflets, and set of commissures adjoining the set of leaflets. The sidewall and each leaflet of the set of leaflets are composed of animal tissue. In addition, a mesh frame is associated with a sidewall of the heart valve.

In an example of a method of implanting a prosthetic valve, a tissue-based valve is delivered to a site of implantation within a recipient. The prosthetic tissue-based valve comprises a sidewall, a set of leaflets, and set of commissures adjoining the set of leaflets. the sidewall and each leaflet of the set of leaflets are composed of animal tissue. A reinforcing tissue structure is secured to the sidewall at the base of a set of leaflets. The reinforcing tissue structure comprises animal tissue having a thickness greater than the thickness of a thickness of the sidewall or the leaflets. The method further comprises securing the prosthetic tissue-based valve at the site of implantation.

In an example of an implantable heart valve device, the valve comprises a conduit formed of animal tissue into a cylindrical shape having a side wall with an inner face and an outer face. The valve further comprises an inner leaflet assembly formed of animal tissue comprising a plurality of leaflets, each leaflet having a cusp edge, a free edge, and a belly. A portion of the cusp edge of each leaflet is connected with a portion of a cusp edge of another leaflet to form a plurality of commissures. The cusp edge of each leaflet of the leaflet assembly is further connected with an inner face of the sidewall. The free edges of leaflet assembly are capable of coapting together.

In an example of a leaflet for use within an implantable heart valve device, the leaflet comprises a sheet of tissue having a free edge connected, a cusp edge, and a belly, the cusp edge contoured in a rounded line. The free edge and the cusp edge are connected at two points forming two corners that are commissure meeting points. Each commissure meeting point is for attachment with at least one other leaflet to form a commissure. Each commissure meeting point incorporates thickened tissue on an outflow face of the sheet of tissue.

In an example of a method of implanting a prosthetic valve, a tissue-based valve is delivered to a site of implantation within a recipient. The tissue-based valve comprises a conduit formed of animal tissue into a cylindrical shape having a side wall with an inner face and an outer face. The tissue-based valve further comprises an inner leaflet assembly formed of animal tissue comprising a plurality of leaflets, each leaflet having a cusp edge, a free edge, and a belly. A portion of the cusp edge of each leaflet is connected with a portion of a cusp edge of another leaflet to form a plurality of commissures. The cusp edge of each leaflet of the leaflet assembly is further connected with an inner face of the sidewall. The free edges of leaflet assembly are capable of coapting together. The method further comprises securing the prosthetic tissue-based valve at the site of implantation.

### BRIEF DESCRIPTION OF THE DRAWINGS

The description and claims will be more fully understood with reference to the following figures, which are presented as exemplary embodiments of the invention and should not be construed as a complete recitation of the scope of the invention.

FIG. 1 provides a perspective view illustration of an embodiment of a tissue-based heart valve with a reinforcing tissue structure.

FIG. 2 provides a side view illustration of an embodiment of a tissue-based heart valve with a reinforcing tissue structure.

FIG. 3 provides a top view illustration of an embodiment of a tissue-based heart valve with a reinforcing tissue structure.

FIG. 4 provides a perspective exploded view illustration of an embodiment of a tissue-based heart valve with a reinforcing tissue structure.

FIG. 5 provides a perspective cut-out view illustration of an embodiment of a reinforcing tissue structure with wire.

FIG. 6 provides a perspective exploded view illustration of an embodiment of a reinforcing tissue structure with wire.

FIG. 7 provides a perspective view illustration of an embodiment of a tissue-based heart valve with a reinforcing tissue structure and a base tissue structure.

FIG. 8 provides a perspective exploded view illustration of an embodiment of a tissue-based heart valve with a reinforcing tissue structure and a base tissue structure.

FIG. 9 provides a perspective view illustration of an embodiment of a tissue-based heart valve with a reinforcing tissue structure, wire mesh structure, and base tissue attachment point.

FIG. 10 provides a perspective exploded view illustration of an embodiment of a tissue-based heart valve with a reinforcing tissue structure, wire mesh structure, and base tissue attachment point.

FIG. 11 provides a front elevation view illustration of the inner side of an embodiment of a leaflet with optional side and/or top tabs.

FIG. 12 provides a perspective view illustration of an embodiment of three leaflets assembled together.

FIG. 13 provides a top-down view illustration of an embodiment of three leaflets assembled together.

FIG. 14 provides a perspective view illustration of the attachment point between two leaflets utilizing rolled tissue in accordance with an embodiment.

FIG. 15 provides a top-down view illustration of the attachment point between two leaflets utilizing rolled tissue in accordance with an embodiment.

FIG. 16 provides a perspective view illustration of an embodiment of a valve having three inner leaflets within a conduit.

FIG. 17 provides a cut-out perspective view illustration of an embodiment of a valve having three inner leaflets within a conduit.

FIG. 18 provides a top-down view illustration of an embodiment of a valve having three inner leaflets within a conduit, the leaflets in a closed position.

FIG. 19 provides a top-down view illustration of an embodiment of a valve having three inner leaflets within a conduit, the leaflets in an open position.

FIG. 20 provides a perspective view illustration of an embodiment of a valve having three inner leaflets within a conduit, the conduit having folded tissue on the inflow and outflow edges.

FIG. 21 provides a front elevation view illustration of an embodiment of a valve having three inner leaflets within a conduit, the conduit having folded tissue on the inflow and outflow edges.

FIG. 22 provides a perspective view illustration of an embodiment of a valve having three inner leaflets within a conduit, the conduit having multiple layers of tissue.

FIG. 23 provides a perspective view illustration of an embodiment of a valve having three inner leaflets within a conduit, the conduit having folded tissue on the inflow edge and portions removed on the outflow edge.

FIG. 24 provides a front elevation view illustration of an embodiment of a valve having three inner leaflets within a conduit, the conduit having folded tissue on the inflow edge and portions removed on the outflow edge.

FIG. 25 provides an illustration of an embodiment to assemble a tissue-based valve with a reinforcing tissue structure.

### DETAILED DESCRIPTION

Turning now to the drawings, devices and methods to provide reinforced support to tissue heart valves are described. Several devices are directed towards a reinforcing tissue-based valves. A reinforced tissue-based valve, in accordance with various devices as described, has the sturdiness and rigidity to withstand stresses that occur in the vasculature, where the forces related to systole and diastole pressures are strong and repetitive. In many instances, a reinforced tissue-based valve prevents and/or mitigates the valve from collapsing. In some instances, a reinforced tissue-based valve maintains shape within the vasculature after implantation.

In numerous variations of devices, a reinforced tissue-based valve incorporates thickened tissue at various locations on the valve. A number of animal tissues can be used to construct a reinforced tissue-based valve, including (but not limited to) bovine pericardium, porcine pericardium, equine pericardium, and human tissue (e.g., human tissue grown in vitro). In many instances, thickened tissue is incorporated at the base of the valve leaflets.

In many variations of devices, a reinforced tissue-based valve incorporates a set of inner leaflets that are associated within a conduit. In several instances, the attachments between the inner leaflets and conduit are reinforced utilizing thickened tissue. In some instances, the conduit wall is reinforced utilizing thickened tissue. In some instances, a filler is utilized within the thicken tissue.

Several variations of devices incorporate a reinforcing tissue structure that further incorporates a mesh of a biocompatible metal or metal alloy, including (but not limited to) nitinol, stainless steel, cobalt-chromium alloys, titanium, and titanium alloys. In various devices incorporating metallic mesh within a reinforcing tissue structure, the mesh further provides sturdiness and rigidity to the reinforcing tissue structure. In a number of reinforcing tissue structures incorporating a mesh, the mesh is encapsulated within the tissue such that the mesh is not exposed.

In many variations valve devices, a tissue-based valve incorporates a mesh frame within the sidewalls of the valve. In several instances, a mesh frame is composed of a biocompatible metal or metal alloy, including (but not limited to) nitinol, stainless steel, cobalt-chromium alloys, titanium, and titanium alloys. In various tissue-based valves incorporating mesh frame within the sidewalls, the mesh frame further provides sturdiness and rigidity to the valve. In a number of tissue-based valves incorporating a mesh frame within the sidewalls, the mesh frame is encapsulated within the tissue of the sidewalls such that the mesh frame is not exposed.

In several variations of tissue-based valves utilizing a mesh frame within the sidewalls, a base stent frame is utilized to dock and situate the valve. In some instances, a base stent further provides sturdiness and rigidity to a tissue valve. In numerous instances, a base stent provides support to the commissures of a tissue valve. In some instances, a base stent is encapsulated in tissue.

Many variations of a tissue-based valve are expandable such that the valve can be compacted and incorporated into a transcatheter delivery system. In some instances, a tissue-based valve is delivered via transcatheter in a transfemoral or transapical approach. In some instances, a balloon or a self-expanding frame is utilized to expand an unexpanded tissue-based valve at the site of insertion.

The described devices, systems, and methods should not be construed as limiting in any way. Instead, the present disclosure is directed toward all novel and nonobvious features and aspects of the various disclosed devices, systems and methods, alone and in various combinations and sub-combinations with one another. The disclosed methods, systems, and devices are not limited to any specific aspect, feature, or combination thereof, nor do the disclosed methods, systems, and apparatus require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatuses can be used in conjunction with other systems, methods, and apparatus.

### Tissue-Based Heart Valves Reinforced with a Tissue Structure

Several devices of the disclosure are directed towards a reinforced tissue-based heart valve. A reinforcing tissue structure can be incorporated within a tissue-based valve, which can provide sturdiness and rigidity to withstand stresses that occur within the vasculature, where the forces related to systole and diastole pressures are strong and repetitive (e.g., at the aortic root). In many instances, a tissue structure prevents and/or mitigates a tissue heart valve from collapsing. In some instances, a tissue structure helps a tissue heart valve maintain shape within the aortic root after implantation.

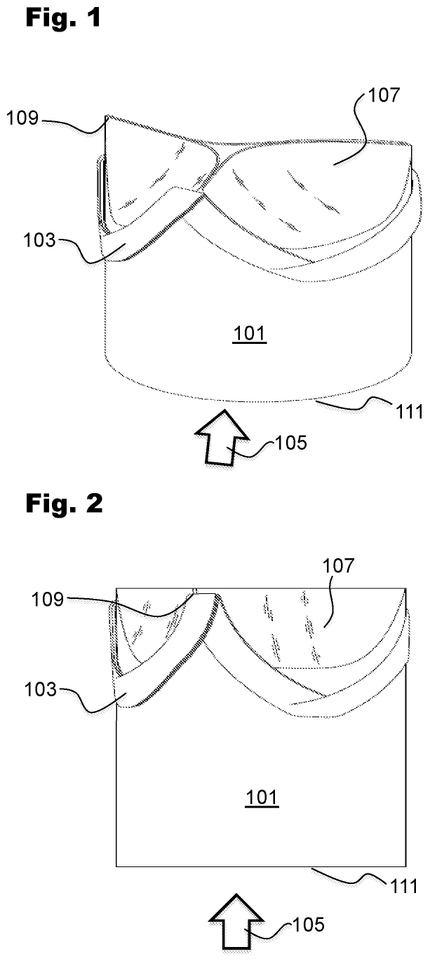

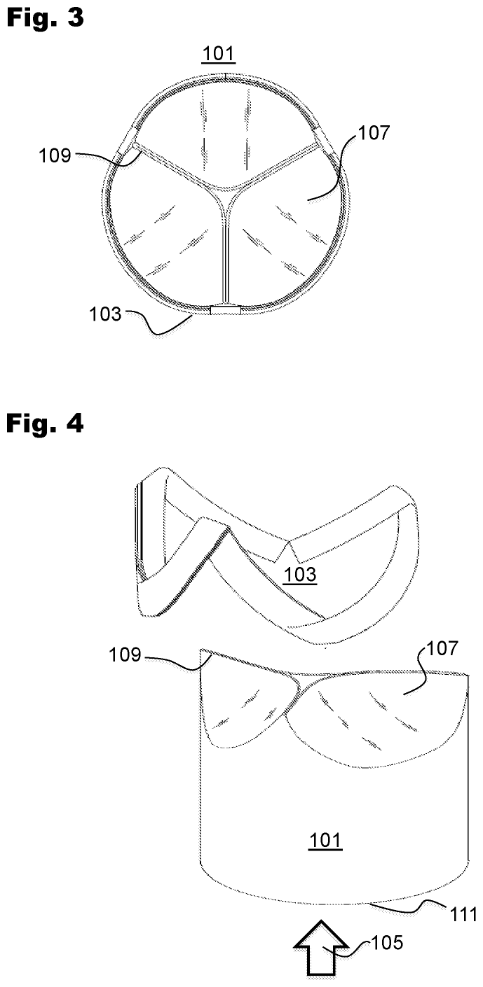

Provided in FIG. 1 is a perspective view, in FIG. 2 is a side view, in FIG. 3 is a top view and in FIG. 4 is a perspective exploded view of an exemplary tissue-based heart valve 101 having an attached reinforcing tissue structure 103. The heart valve 101 and attached reinforcing tissue structure 103 can be utilized as a heart valve replacement to treat heart valve disease. Numerous variations of devices are directed to tissue heart valves to replace dysfunctional aortic valves; however, it should be understood that the mitral valve, tricuspid valve, and pulmonary valve can also be replaced. Blood flow through the heart valve is depicted by arrow 105.

As can be seen in figures, the exemplary tissue-based heart valve 101 has three leaflets 107 that are composed of tissue that extend from a tissue-based sidewall 108. The leaflets are joined and/or abut at the side commissures 109. In some instances, leaflets are adjoined together at the commissures. Adjoining leaflets at the commissures can be done by any appropriate means, including (but not limited to) sutures, staples, and/or biocompatible adhesive. Typically, two or three leaflets are formulated in a tissue-based heart valve, but it should be understood that the number of leaflets can vary and still fall within various variations of tissue-based valves of the disclosure. Further, as can be seen in FIGS. 1 through 4, the valve sidewall and leaflets can have a unibody tubular design, meaning the set of leaflets 107 and sidewall 113 are formed from the same cut of tissue. Two ends of a sheet of tissue can be adjoined to form a cylindrical sidewall, and the leaflets are folded inward along the top edge of the cylindrical sidewall. Adjoining two ends of a sheet of tissue can be done by any appropriate means, including (but not limited to) sutures, staples, and/or biocompatible adhesive.

When replacing an aortic valve, a tissue-based valve 101 can be situated within the aortic root such that the base 111 is located at the aortic annulus, the top of the leaflets is located at the sinotubular junction, and blood flow follows arrow 105 (e.g., from left ventricle into ascending aorta).

A number of tissue-based valves utilize animal tissue to form the valve and/or a reinforcing tissue structure. Animal tissue that can be used include (but are not limited to) bovine pericardium, porcine pericardium, equine pericardium and human tissue (e.g., transplanted human pericardium, or human tissue grown in vitro). In some instances, regenerative tissue is utilized to form tissue portions of a tissue-based heart valve, including leaflets. In some instances, a regenerative tissue is grown in vitro prior to implantation in accordance with methods as understood in the art. For more detailed discussion on regenerative heart valve tissue, see the description described within the section labeled “Regenerative Tissue,” which is provided herein.

In a number of instances, a tissue-based heart valve is to be inserted into an aortic root to replace a dysfunctional aortic valve, where the forces related to systole and diastole pressures are strong and repetitive. Because tissue-based heart valves are generally composed of soft tissue, they lack sufficient rigidity to withstand strong pulsatile pressures in the aortic root and elsewhere. The pressures can cause an implanted tissue-based heart valve to collapse, causing great damage and preventing the valve from properly integrating within an aortic root. Accordingly, several tissue-based valves of the instant disclosure incorporate a reinforcing tissue structure that provides structural rigidity capable of withstanding constricting and pulsatile forces associated with blood pressure in the aortic root or elsewhere. In many instances, a reinforcing tissue structure helps maintain a tissue-based heart valve's shape and functionality while under stress from the blood pressure forces.

As depicted in an embodiment in FIGS. 1 to 4, a reinforcing tissue structure 103 can be incorporated onto a heart valve, and in some instances, tissue structure 103 is incorporated at the cusp edge of the leaflets 107. Tissue structure 103 can have a width and a length. The width of a tissue structure can vary, and in some instances, the width is between one-tenth and one-half the height of the tissue-based valve's sidewall. In various instances, the width of a tissue structure is one-tenth, one-eight, one-sixth, one-fifth, one-fourth, one-third, or one-half the height of the tissue-based valve's sidewall. In some instances, the length of a tissue structure is long enough to encircle the tissue-based sidewall. In some instances, the tissue structure contours along the cusp edge of each leaflet and up to each leaflet commissure, while encircling the sidewall.

A reinforcing tissue structure can provide rigidity and support to a tissue-based heart valve. In some instances, a reinforcing tissue structure is able to support a tissue-based heart valve to withstand the forces within an aortic root such that the heart valve can maintain a valvular shape. Accordingly, in some instances, a reinforcing tissue structure has enough compressive strength to prevent collapse of a regenerative heart valve due to constricting forces within the aortic root. Likewise, in some instances, a reinforcing tissue structure has enough fatigue strength such that a regenerative heart valve is able to withstand pulsatile pressures associated with systole and diastole. As known in the art, pressures within aortic root can be approximately 120 systolic mmHg in a typical human, and can reach above 150 systolic mmHg or even 180 systolic mmHg in an individual suffering from severe hypertension. Accordingly, in various instances, a tissue-based heart valve is able to withstand pressures of at least about 100 mmHg, 110 mmHg, 120 mmHg, 130 mmHg, 140 mmHg, 150 mmHg, 160 mmHg, 170 mmHg, or 180 mmHg.

In several instances, a reinforcing tissue structure is a thickened tissue, having a thickness greater than the leaflets and/or valve sidewall. In some instances, a reinforcing tissue structure has a thickness of about 1.5×, 2×, 2.5×, 3×, 4×, 5× or greater than 5×, as compared to the tissue thickness of the leaflets and/or valve sidewall. In some instances, a reinforcing tissue structure is a tissue that has been folded to create the thickness. In some instances, a reinforcing tissue structure is tissue that has been layered, with each layer attached to its proximate layer, to create the thickness. In some instances, a reinforcing tissue structure is tissue that has been stuffed with a biocompatible filler to create the thickness. In some instances, a reinforcing tissue structure is tissue that has been grown to a greater thickness.

A reinforcing tissue structure can be secured to the base of the leaflets and leaflet commissures. In some instances, a reinforcing tissue structure is secured to the base of the leaflets using sutures. In some embodiments, sutures used to secure a reinforcing tissue structure are bio-absorbable. In some instances, a reinforcing tissue structure is secured to the base of the leaflets using a biocompatible adhesive.

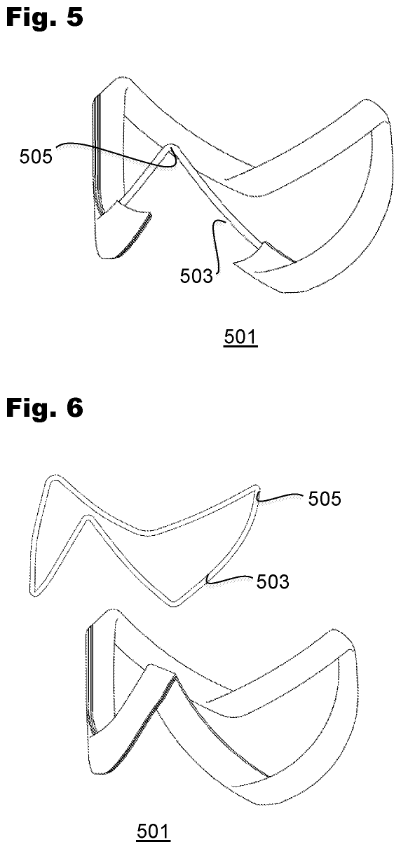

Provided in FIG. 5 is a perspective cut-out view and in FIG. 6 is a perspective exploded view of a reinforcing tissue structure 501 incorporating a wire 503 within the tissue. A wire can further provide sturdiness and rigidity to the tissue-based valve.

A wire can provide support to the commissures of a tissue-based heart valve. As shown in FIGS. 5 and 6, the wire 503 has three upward protruding (i.e., in the direction of blood flow) corners 505. The corners are able crimp onto commissures (i.e., the abutting portions of the leaflets). Accordingly, a wire can also support and maintain the shape of the commissures, while still allowing the leaflets to function (i.e., open and close the valve leaflets based on systole and diastole).

In several instances, a wire is composed of a biocompatible metal or metal alloy, including (but not limited to) nitinol, stainless steel, cobalt-chromium alloys, titanium, and titanium alloys. In some instances, a wire is situated internally within a reinforcing tissue structure. In some instances, a wire is encapsulated within a reinforcing tissue structure such that the wire is not exposed. In some instances, a wire is attached externally to a reinforcing tissue structure. In some instances, a wire is situated in between the reinforcing tissue structure and a sidewall of a tissue valve. In some instances, a wire is in between a reinforcing tissue structure and a sidewall of a tissue valve such that the wire is not exposed.

The tissue of a reinforcing tissue structure can be derived from any appropriate tissue source. Animal tissue can be utilized to form a reinforcing tissue structure, including (but not limited to) bovine pericardium, porcine pericardium, equine pericardium, and human tissue (e.g., transplanted human pericardium, or human tissue grown in vitro). In some instances, regenerative tissue is utilized to form a reinforcing tissue structure that incorporates a wire.

A wire can be secured to a reinforcing tissue structure using sutures. In some instances, sutures used to secure a wire are bio-absorbable. In some instances, a wire is secured to a reinforcing tissue structure using a biocompatible adhesive.

A reinforcing tissue structure that incorporates a wire can be grown in vitro in the presence of the wire such that the reinforcing tissue structure grows around and within the metallic wire to encase it. In some instances, a reinforcing tissue structure is layered around a wire and sutured to encase the metallic mesh. In some instances, a reinforcing tissue structure is folded around a wire and sutured to encase the metallic wire. In some instances, a wire is sutured onto an external portion of a reinforcing tissue structure.

### Tissue-Based Heart Valves Incorporating a Base Tissue Structure

A tissue-based heart valve can incorporate a base tissue structure, which can be incorporated in addition to, or without, a reinforcing tissue structure. A base tissue structure can support a tissue valve tissue-based heart valve from the stresses that occur within the aortic root, where the forces related to systole and diastole pressures are strong and repetitive. A base tissue structure can prevent and/or mitigate the tissue-based heart valve from collapsing. A base tissue structure can help the tissue-based heart valve maintain shape within the aortic root after implantation.

In several instances, a base structure is utilized to dock and situate the tissue valve at the site of deployment. In some instances, a base tissue structure provides support to the lower end of a tissue valve. In some instances, a base tissue structure provides a protruding structure to secure a tissue-based valve to the annulus of an aortic root. In some instances, sutures are used to secure a protruding base structure of a tissue valve to the aortic root. In some instances, a protruding base structure of a tissue valve is secured to the aortic root using a biocompatible adhesive.

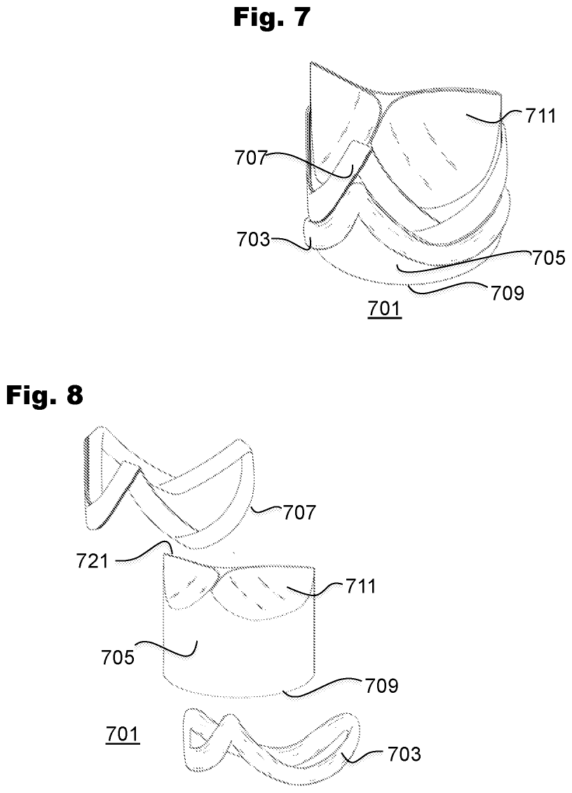

Provided in FIG. 7 is a perspective view and in FIG. 8 is a perspective exploded view of a tissue-based heart valve 701 incorporating a base tissue structure 703 to support the sidewall 705 of valve 701 and provide a protruding structure for attachment in an individual's aortic root. In these figures, the base tissue structure 703 is rolled tissue to provide some girth and is attached to the sidewall 705. In many embodiments, the thickness of the base tissue structure can vary, but should provide enough thickness such that it can be utilized to provide a means of attachment within an individual's aortic root. In some embodiments, the base tissue structure 703 has a width that extends from the base of the reinforcing tissue structure 707 to near or at the inflow end of the tissue valve 709. It should be understood that the edge of the inflow end 709 can be contoured to meet any appropriate shape. In some embodiments, the edge of the inflow end is scalloped such that the edge follows along the contours the base tissue structure. In some instances, the tissue-based heart valve 701 is also supported by a reinforcing tissue structure 707, which is attached to the heart valve at the base of the leaflets 711.

The width of a base tissue structure can vary, and in some instances, the width is between one-tenth and one-half the height of the tissue-based valve's sidewall. In various instances, the width of a base structure is one-tenth, one-eight, one-sixth, one-fifth, one-fourth, one-third, or one-half the height of the tissue-based valve's sidewall. In some instances, the length of a base structure is long enough such that the tissue structure is situated along the edge of the reinforcing tissue structure 707, contouring along the reinforcing tissue structure.

In some instances, a base tissue structure is a thickened tissue, having a thickness greater than the tissue thickness of the leaflets and/or valve sidewall. In some instances, a base tissue structure has a thickness of about 1.5×, 2×, 2.5×, 3×, 4×, 5× or greater than 5×, as compared to the thickness of the leaflets and/or valve sidewall. In some instances, a base tissue structure incorporates a wire, such as (for example) the wire described in FIGS. 5 and 6.

Animal tissue can be utilized to form a base tissue structure. Animal tissues that can be used include (but are not limited to) bovine pericardium, porcine pericardium, equine pericardium, and human tissue (e.g., transplanted human pericardium, or human tissue grown in vitro). In some instances, regenerative tissue is utilized. In some instances, a regenerative tissue is grown in vitro prior to implantation in accordance with methods as understood in the art. For more detailed discussion on regenerative heart valve tissue, see the description described within the section labeled “Regenerative Tissue,” which is provided herein.

A base tissue structure can be secured to the sidewall of the tissue-based valve. In some instances, a base tissue structure is secured to the sidewall of the tissue-based valve using sutures. In some instances, sutures used to secure a base tissue structure are bio-absorbable. In some instances, a base tissue structure is secured to the base of the sidewall using a biocompatible adhesive.

### Tissue-Based Heart Valves Incorporating a Metallic Frame

A number of devices are directed to tissue-based heart valves that incorporate a mesh frame within the sidewall of the heart valve. A mesh frame supports a tissue-based valve from the stresses that occur within the aortic root, where the forces related to systole and diastole pressures are strong and repetitive. A mesh frame can help prevent and/or mitigate collapsing of the tissue-based valve. Further, a mesh frame can help a tissue-based valve maintain shape within the aortic root after implantation.

A mesh frame can be composed of a biocompatible metal or metal alloy, including (but not limited to) nitinol, stainless steel, cobalt-chromium alloys, titanium, and titanium alloys. The mesh frame can further provide sturdiness and rigidity to the tissue-based valve.

A mesh frame can be incorporated within the sidewall of a tissue valve. In some instances, the mesh frame is encapsulated within the tissue of the sidewall such that the mesh is not exposed. In some instances, a mesh frame is positioned externally to a tissue heart valve surrounding the sidewall of the valve. In some instances, a mesh frame is attached to the external side of the sidewall of a tissue heart valve. In some instances, a metallic mesh frame is attached to the internal side of the sidewall of a tissue heart valve. In some instances, metallic mesh frame is secured to the sidewall of a tissue-based valve using sutures. In some instances, sutures used to secure a metallic mesh frame are bio-absorbable. In some instances, a metallic mesh frame is secured to a side-wall of a tissue-based valve using a biocompatible adhesive.

When utilizing a mesh frame secured to the sidewall of a tissue valve, a base stent can be utilized to dock and situate the tissue valve. A base stent can further provide sturdiness and rigidity to a tissue valve. A base stent can provide support to the commissures of a tissue valve. A base stent can be encapsulated in tissue.

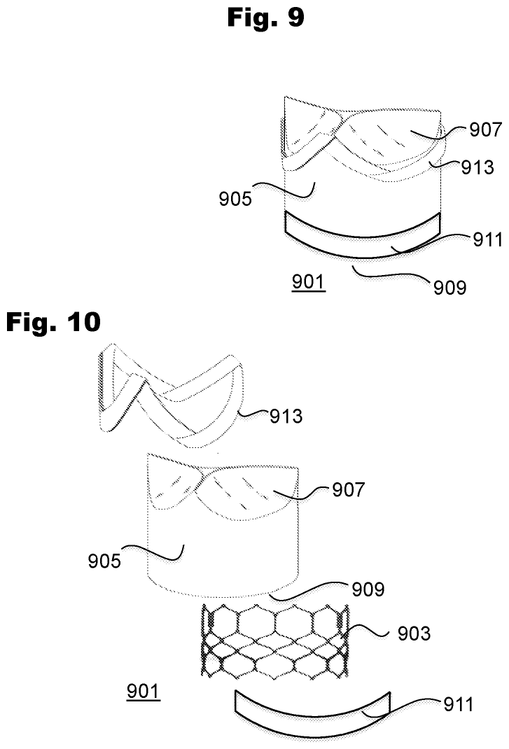

Provided in FIG. 9 is a perspective view and in FIG. 10 is a perspective exploded view of a tissue-based heart valve 901 incorporating a mesh frame 903 to support the sidewall 905 of the valve. In these figures, the mesh frame 903 is encapsulated within the sidewall 905, however, a mesh frame can be attached to or situated adjacent to the sidewall, internally or externally. The height of the mesh frame 903 can vary, but in some instances, the upper edge of mesh frame 903 will be incorporated from some point below the leaflets 907 and the mesh will extend to the inflow end of the tissue valve 909. In some instances, the upper edge of mesh frame 903 will extend from the base of the leaflets 907 to the inflow end of the tissue valve 909. In some instances, a tissue flap 911 is utilized at the base of the sidewall of a tissue-based heart valve 901 incorporating a mesh frame, which can be used to help secure the tissue-based heart valve within the aortic root of an individual.

A tissue flap can have a width and length. The width of a tissue flap can vary, and in some instances, the width is between one-tenth and one-half the height of the tissue-based valve's sidewall. In various instances, the width of a tissue flap is one-tenth, one-eight, one-sixth, one-fifth, one-fourth, one-third, or one-half the height of the tissue-based valve's sidewall. In some instances, the length of the tissue flap is long enough such that the tissue flap encircles the sidewall. In some instances, the tissue flap is situated along the inflow edge of the tissue-based valve sidewall, contouring along the inflow edge.

A tissue flap can be derived from any appropriate tissue source. Animal tissues that can be used to form a tissue flap include (but are not limited to) bovine pericardium, porcine pericardium, equine pericardium, and human tissue (e.g., transplanted human pericardium, or human tissue grown in vitro). In some instances, regenerative tissue is utilized to form a tissue flap.

A metallic mesh frame can be secured to the sidewall of a tissue-based valve. In some instances, a metallic mesh frame is secured to the sidewall of a tissue-based valve using sutures. In some instances, sutures used to secure a metallic mesh frame are bio-absorbable. In some instances, a metallic mesh frame is secured to a tissue-based valve using a biocompatible adhesive.

Likewise, a tissue flap can be secured to the sidewall of a tissue-based valve. In several instances, a tissue flap frame is secured to the sidewall of a tissue-based valve using sutures. In some instances, sutures used to secure a tissue flap frame are bio-absorbable. In some instances, a tissue flap is secured to a tissue-based valve using a biocompatible adhesive.

The tissue-based heart valve 901 can also be supported by a reinforcing tissue structure 913, which is attached to the heart valve at the base of the leaflets 907. In some instances, a reinforcing tissue structure is a thickened tissue, having a thickness greater than the tissue thickness of the leaflets and/or valve sidewall. In some instances, a reinforcing tissue structure has a thickness of about 1.5×, 2×, 2.5×, 3×, 4×, 5× or greater than 5×, as compared to the thickness of the leaflets and/or valve sidewall. In some instances, a reinforcing tissue structure incorporates a mesh, such as (for example) the mesh described in FIGS. 5 and 6.

A tissue flap can be used for docking a tissue-based valve, especially valves that incorporate a mesh frame within or along its sidewall. In numerous instances, the inflow end the mesh frame can situate within a tissue flap, and in some instances, the mesh frame is secured to the tissue flap.

In several instances, a base stent is composed of a biocompatible metal or metal alloy, including (but not limited to) nitinol, stainless steel, cobalt-chromium alloys, titanium, and titanium alloys. In some instances, a base stent is encapsulated in tissue such that the metal is not exposed to the local environment.

### Tissue-Based Valves with Inner Leaflet Assembly

Several devices are directed towards tissue-based heart valves that are assembled utilizing a number of leaflets contoured to fit within a conduit. In many instances, a set of leaflets are assembled together to form the leaflet component of a tissue-based heart valve. Any appropriate number of leaflets may be utilized to form a valve, but typically two or three leaflets are utilized, mimicking naturally occurring heart valves. In various instances, a valve has 2, 3, 4, or 5 of leaflets. A set of leaflets can be interconnected to form cusps and commissures and the commissures and lower edge of cusps can be attached to the inner face of the side wall of the conduit, forming leaflets with free edges that are able to open and close to allow unidirectional blood flow through the conduit and preventing backflow.

In many instances, each leaflet for assembly has a shape, having a free edge, a central area (or belly area), a cusp area (or base area), and a commissure area where each leaflet adjoins another leaflet. A leaflet also has two sides, the inflow side and the outflow side. In several instances, a cusp edge is rounded, which can provide the hemodynamic performance desired. In some instances, the free edge of a leaflet has an extended length (longer than the minimum distance between the leaflet's commissures), which can allow for retraction that can occur during a tissue regenerative process (e.g., post-implantation as host tissue regenerates within valve structure). In various instances, the extended length of the free edge is between 1.1× and 2.0×, and particularly is 1.1×, 1.2×, 1.3×, 1.4×, 1.5×, 1.6×, 1.7×, 1.8×, 1.9×, or 2.0×, longer than the minimum distance between the leaflet's commissures. It is to be understood that the minimum distance between the leaflet's commissures is the minimal distance necessary for closure/coaptation of the leaflets resulting in formation of a coaptation zone when a tissue-based valve is closed.

A leaflet can be reinforced at the commissures where leaflets attach to one and another; and/or a leaflet can be reinforced at the cusp edge where leaflets attach to a conduit. In some instances, thickened tissue is provided at commissure and/or at the cusp edge for strength. In some instances, thickened commissure tissue has a thickness of about 1.5×, 2×, 2.5×, 3×, 4×, 5× or greater than 5×, as compared to the thickness of the leaflets. Any appropriate means to provide thickened tissue can be utilized, including (but not limited to) layering tissue, rolling tissue, or folding tissue. In some instances, tissue can be thickened by inserting a filler within layered, rolled, and/or folded tissue. Any appropriate filler can be utilized, especially biocompatible fillers including (but not limited to) nitinol, cobalt-chromium, titanium, stainless steel, poly(lactic-co-glycolic) acid (PLGA), polyvinylchloride (PVC), polyethylene (PE), polypropylene (PP), polytetrafluoroethylene (PTFE), polyurethane (PU), polyethylene terephthalate (PET), polyether sulfone (PES), polyglycolic acid (PGA), polylactic acid (PLA), poly-D-lactide (PDLA), poly-4-hydroxybutyrate (P4HB), and polycaprolactone (PCL). In some instances, a filler is encapsulated within a biocompatible material (e.g., material encapsulated with PET). Furthermore, the cusp edge can be reinforced with a sleeve on the inflow side of the leaflet.

The disclosure is also directed to various assemblies and/or structures that prevent and/or mitigate a leaflet's free edge and belly from contacting an inner wall of a conduit to which the leaflets are attached when the valve is implanted and function. To prevent and/or mitigate contact, leaflets can be secured together at the commissures, preventing these distal portions of the leaflet free edges from opening up when blood flows through the valve. In various instances, a bulky structure is utilized to block the central portions of a leaflet commissure edge from contacting an inner wall of a conduit. In some instances, thickened tissue utilized to strengthen the commissure can also provide bulk to block the central portions of a leaflet free edge from contacting an inner wall of a conduit.

A conduit can be utilized to house a set leaflets to form a valve. A conduit, in accordance with many embodiments, is a tubular prosthetic structure that mimics vasculature structure. A conduit can be formed utilizing a sheet of tissue that is formed into a cylindrical shape and connected at two opposite side edges to form a tube. Any appropriate means to connect two side edges of tissue can be utilized, including (but not limited to) sutures or a biocompatible adhesive. In some instances, portions of the wall of the conduit are removed beyond the leaflets at the distal portion of the conduit wall. Removal of portions of the conduit wall allow coronary access when the valve is implanted in the aortic position, but still maintain a suitable attachment between the set of inner leaflets and conduit. which can create space for effluent blood to access nearby sinuses (e.g., the left and right coronary sinuses at the site of the aortic valve) when implanted.

A set of leaflets can be attached to the inner wall of a conduit, forming a uni-directional flow valve. Accordingly, in various instances, each leaflet has a contoured cusp for attachment to the inner conduit wall. In several instances, each leaflet is attached to the inner wall via attachment points at the commissure and the cusp edge. In some instances, thickened tissue at the commissures help ensure attachment to the conduit wall at the commissure attachment points. In some instances, a rigid and/or solid structure (e.g., 4-hole metal bar) is attached to the outer wall of the conduit at the commissure attachment points to reinforce the attachment between the leaflets and the conduit. An outer rigid and/or solid structure can be in connection with the inner commissure attachment point by sutures, staples, hooks, and/or other appropriate means.

The wall of the conduit can be strengthened with thickened tissue (i.e., conduit wall tissue thicker than the leaflet tissue). In some instances, a thickened conduit wall tissue has a thickness of about 1.5×, 2×, 2.5×, 3×, 4×, 5×, or greater than 5×, as compared to the thickness of the leaflets. A conduit wall can be thickened with a plurality of tissue layers, which can be attached together via sutures, biocompatible adhesive, staples, and/or other appropriate means. In some instances, the inflow and/or outflow edge of the conduit is thickened. Any appropriate means to provide thickened tissue can be utilized to thicken the inflow or outflow edge, including (but not limited to) layering tissue, rolling tissue, or folding tissue. In some instances, tissue can be thickened by inserting a filler within layered, rolled, and/or folded tissue. Any appropriate filler can be utilized, especially biocompatible fillers, including (but not limited to) nitinol, cobalt-chromium, titanium, stainless steel, poly(lactic-co-glycolic) acid (PLGA), polyvinylchloride (PVC), polyethylene (PE), polypropylene (PP), polytetrafluoroethylene (PTFE), polyurethane (PU), polyethylene terephthalate (PET), polyether sulfone (PES), polyglycolic acid (PGA), polylactic acid (PLA), poly-D-lactide (PDLA), poly-4-hydroxybutyrate (P4HB), and polycaprolactone (PCL). In some instances, a filler is encapsulated within a biocompatible material (e.g., material encapsulated with PET).

A prosthetic valve can include radiopaque structures attached upon and/or within a valve, which may help visualize the valve when viewed utilizing radiography, especially during implantation and/or check-up procedures. Any appropriate radiopaque structures can be utilized, such as (for example) metals and metal alloys. In many instances, a radiopaque structure is situated on or near a valve structure or feature such that the valve structure or feature can be identified via radiography. For example, in some instances, a radiopaque structure is situated on or near the commissure connecting points, enabling their visualization. In some instances, a 4-hole metal bar is utilized to provide radiopaque identification of commissure connecting points. Radiopaque sutures for assembling the valve can also be used to provide visualization.

Animal tissue and/or a biocompatible polymer can be utilized to form a leaflet and/or conduit of a tissue-based heart valve and/or a reinforcing tissue structure, including (but not limited to) bovine pericardium, porcine pericardium, equine pericardium, and human tissue (e.g., transplanted human pericardium, or human tissue grown in vitro). In some instances, regenerative tissue is utilized to form tissue portions of a tissue-based heart valve, including leaflets and/or conduit. In some instances, a regenerative tissue is grown in vitro prior to implantation in accordance with methods as understood in the art. For more detailed discussion on regenerative heart valve tissue, see the description described within the section labeled “Regenerative Tissue,” which is provided herein. Likewise, any appropriate biocompatible polymer may be utilized to form the leaflets and/or conduit, including (but not limited to) poly(lactic-co-glycolic) acid (PLGA), polyvinylchloride (PVC), polyethylene (PE), polypropylene (PP), polytetrafluoroethylene (PTFE), polyurethane (PU), polyethylene terephthalate (PET), polyether sulfone (PES), polyglycolic acid (PGA), polylactic acid (PLA), poly-D-lactide (PDLA), poly-4-hydroxybutyrate (P4HB), and polycaprolactone (PCL).

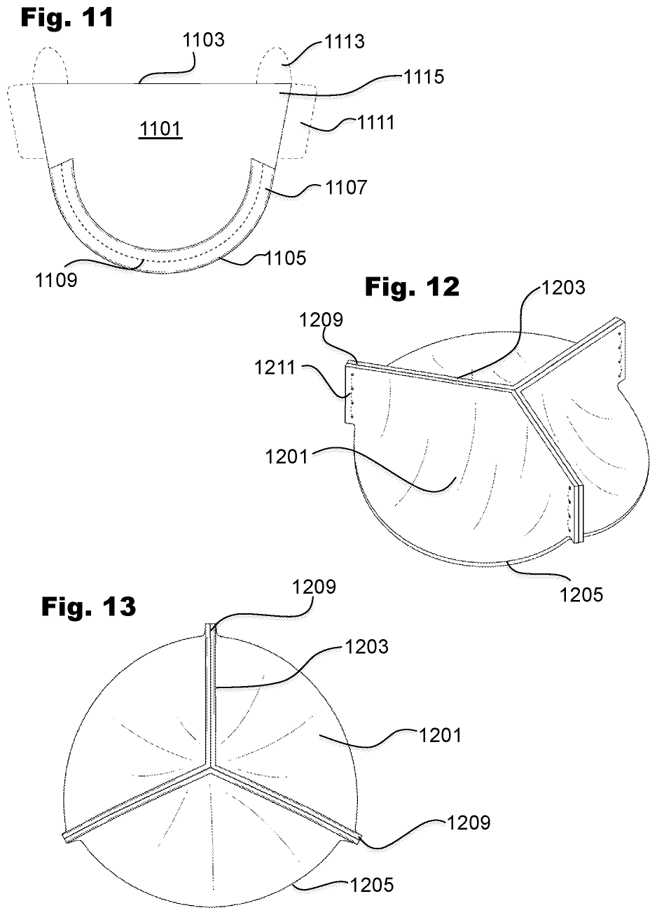

Provided in FIG. 11 is an example of a leaflet 1101 for assembly into a valve in which the inflow side is visible. The leaflet 1101 has a free edge 1103 and rounded cusp edge 1105. The rounded cusp edge 1105 provides the desired hemodynamic performance of the valve. At the distal ends of the free edge are commissure meeting points 1115, where a leaflet can attach to another leaflet.

Along the rounded cusp edge 1105 is an optional sleeve 1107 attached thereupon the inflow side of the leaflet. The sleeve 1107 can provide reinforcement to the rounded cusp edge 1105 and/or provide more tissue for attachment to the inner wall of a circular conduit. The sleeve 1107 can be attached by any appropriate means, including (but not limited to) sutures, staples, and/or biocompatible adhesive. In this example, the sleeve 1107 is attached to the rounded cusp edge 1105 via sutures 1109.

The leaflet 1101 can optionally include tissue tabs, such as the side tabs 1111 and top tabs 1113 depicted in FIG. 11. Side tabs 1111, as can be seen, are extensions of tissue extending from a portion of the cusp edge 1105 proximal to a commissure location 1115, whereas top tabs 1113 are extensions of tissue extending from a portion of the free edge 1103 proximal to the commissure location 1115. Tissue tabs can be utilized to reinforce the leaflet 1101 at the commissure meeting points 1115 by folding or rolling the tissue tabs onto the outflow face by the commissure meeting points. Alternatively, detached tissue pieces can be layered onto the commissure 1115 of the leaflet 1101 to provide reinforcement. As described herein, a filler can be incorporated within the layered, rolled, and/or folded tissue reinforcement at the commissures 1115 of the leaflet 1101.

Provided in FIGS. 12 and 13 are perspective views and of the outflow side of a leaflet assembly 1201 in which three leaflets are assembled together. Each leaflet has a free edge 1203 and a rounded cusp edge 1205, and is portrayed with rounded cusp edges 1205 curved outward as if it were in contact with the inner wall of a circular conduit. Along the rounded cusp edge 1205 of each leaflet 1201, an optional sleeve (not shown) can be attached thereupon on the inflow side of the leaflets.

The three leaflets of the assembly 1201 are adjoined such that commissures 1209 are formed at the distal ends of free edges 1203. Each leaflet 1201 is attached to the other two leaflets at the commissure 1209, resulting in three attachment points between the three leaflets are formed. As shown, the commissures 1209 are attached together via sutures 1211, however, it should be understood that any appropriate means of attachment can be utilized, including (but not limited to) sutures, staples, and/or biocompatible adhesive. When the leaflets are in closed position (such as depicted in FIGS. 12 and 13), the free edge of each leaflet 1203 coapts. Although three leaflets are shown assembled together in this particular assembly, any number of leaflets as described herein can be utilized.

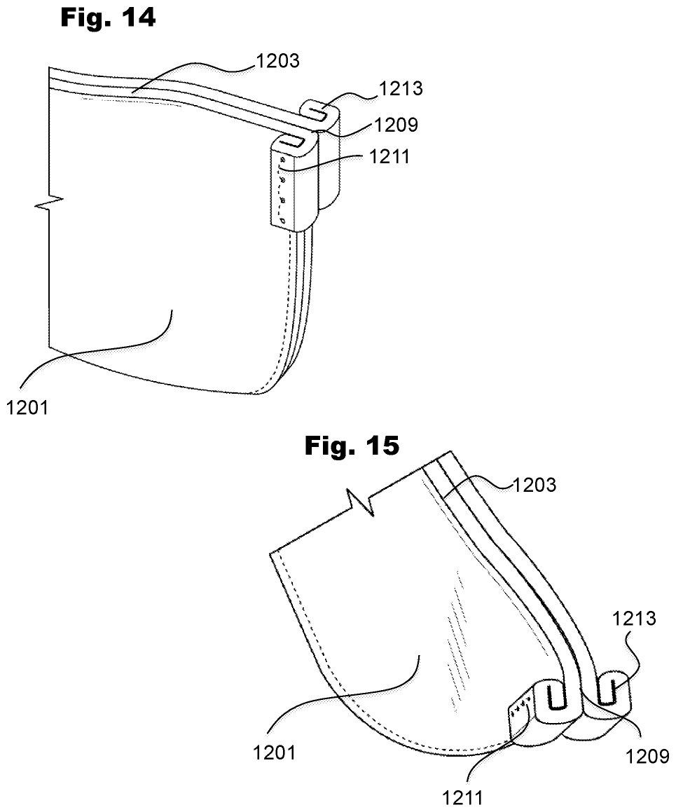

FIGS. 14 and 15 are perspective and top-down views zoomed onto the region of where two leaflets 1201 are adjoined together to form the commissure 1209. These figures further depict reinforced tissue 1213 at the commissure 1211. In particular, these figures depict rolled tissue from a side tab, although it should be understood that reinforced tissue could be layered, folded, and/or rolled, and it is to be further understood that reinforced tissue could be provided from a top tab in addition or in lieu of the side tab. Reinforced tissue can provide strength at the commissures 1209. The reinforcing tissue can help strengthen the connection between two leaflets 1201 and/or the connection between a commissure 1209 and an inner wall of a conduit. Although sutures 1211 are shown to attach to the commissure 1209, it should be understood that any appropriate means of attachment can be utilized, including (but not limited to) sutures, staples, and/or biocompatible adhesive.

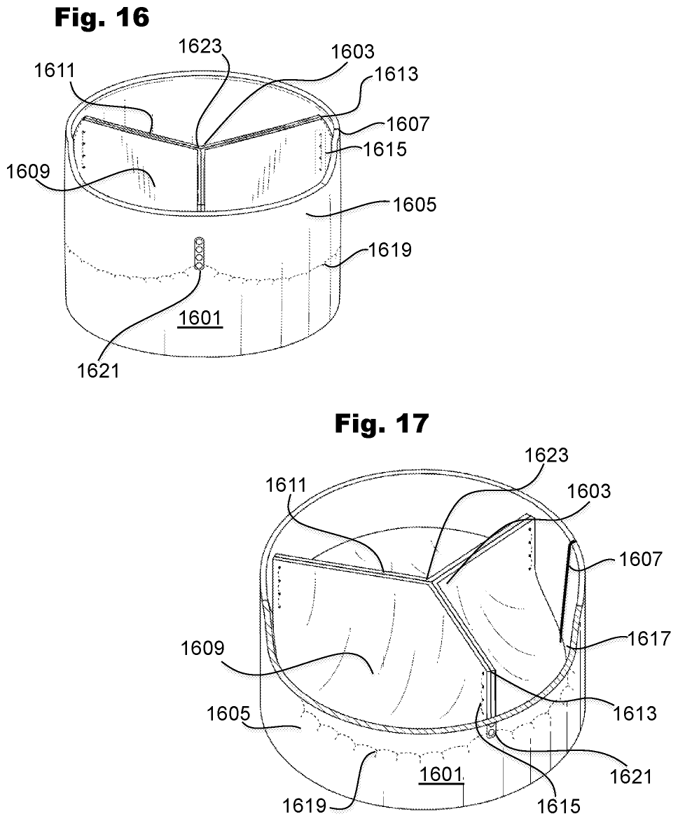

FIG. 16 provides a perspective view of an example of a tissue-based valve 1601 having a set of inner leaflets (1603) attached within a conduit (1605). FIG. 17 provides a cut-out view of the tissue-based valve 1601 in which a portion of the conduit 1605 wall is visually removed such that the inner leaflets 1603 and their association with the conduit (1605) is viewable.

In this example, the conduit 1605 is a sheet of tissue with two ends adjoined together 1607 to form a tubular structure to house the set of inner leaflet assembly 1603. Any appropriate means can be utilized to adjoin the two sides of the sheet of tissue, including (but not limited to) sutures, staples, and/or biocompatible adhesive.

Three individual leaflets 1609 are attached together to form the inner leaflet assembly 1603. The three leaflets 160) are assembled such that a free edge 1611 is adjacent with a free edge of another leaflet, forming a coaptation zone 1623. Each leaflet 1609 is attached to the other two leaflets at the commissure 1613, such that three attachment points between the three leaflets are formed. As shown, the commissures 1613 are attached together via sutures 1615, however, it should be understood that any appropriate means of attachment can be utilized, including (but not limited to) sutures, staples, and/or biocompatible adhesive. Likewise, the commissures 1613 and the rounded cusp edge 1617 are attached to the inner wall of the conduit 1605 via sutures 1619, however, it should be understood that any appropriate means of attachment can be utilized, including (but not limited to) sutures, staples, and/or biocompatible adhesive. As shown in this embodiment, a 4-hole bar 1621 is utilized as a rigid/solid structure to reinforce the attachment between the commissures 1613 of the leaflets and conduit wall. It is noted, however, that sutures alone or any other appropriate rigid/solid structure can be utilized to help reinforce this attachment. The commissures 1613 can also incorporate reinforced tissue (not shown) such as layered, rolled, and/or folded tissue, as detailed in the description of FIGS. 14 and 15. In addition, the rounded cusp can also include a sleeve (not shown) on the inflow side of the leaflets to help reinforce attachment between the rounded cusp edge 1617 and the conduit wall. Although three leaflets are shown assembled together in this particular embodiment, any appropriate number of leaflets can be utilized.

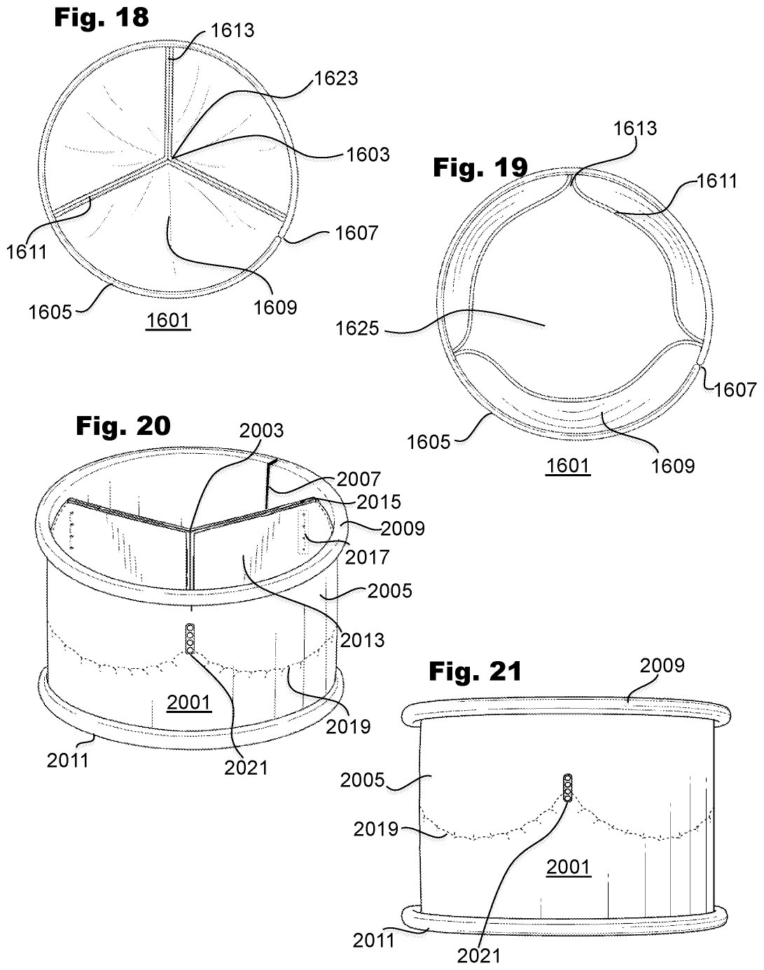

FIGS. 18 and 19 each provide a top-down view of the outflow portion of a tissue-based valve 1601 having an inner leaflet assembly 1603 attached within a conduit 1605 with FIG. 18 depicting the valve 1601 in a closed position and FIG. 19 depicting the valve 1601 in an open position. The three leaflets 1607 are adjoined such each leaflet is attached to the other two leaflets at the commissures 1613.

When the valve is closed, the three leaflets meet at the coaptation zones 1623 which prevents regurgitant blood flow back through the valve 1601.

When the valve is open, the coaptation zones 1623 of the leaflet separate, forming an aperture 1625, which allows blood to flow through the valve. The free edges 1611 and the belly area of the leaflets are prevented from contacting the inner side of the conduit wall, which can be prevented by utilizing a bulky structure such as reinforcing structures at the commissures 1613.

FIGS. 20 and 21 depict an example of a tissue-based valve 2001 in perspective and front-elevation views, respectively. The tissue-based valve 2001 incorporates inner leaflets 2003 within a conduit 2005. In this example, the conduit 2005 is a sheet of tissue with two sides adjoined together 2007 to form a tubular structure to house the inner leaflet assembly 2003. Any appropriate means can be utilized to adjoin the two sides of the sheet of tissue, including (but not limited to) sutures, staples, and/or biocompatible adhesive. The conduit 2005 incorporates reinforced tissue along the outflow edge 2009 and inflow edge 2011. In particular, these figures depict a conduit 2005 with folded tissue at these edges, although it should be understood that reinforced tissue could be layered, folded, and/or rolled. Reinforced tissue can provide strength at the outflow edge 2009 and/or the inflow edge 2011. In addition, the reinforce tissue can help strengthen the eventual connection between the tissue-based valve 2001 to the local tissue at the site of implantation (e.g., within the recipient's vasculature). Folded, rolled, and/or layered tissue may include a biocompatible filler, as described herein.

The inner leaflet assembly 2003 of the tissue-based valve 2001 depicted in FIGS. 20 and 21 can be constructed and attached in a similar manner to the inner leaflet assembly portrayed in FIGS. 16 and 17. Three individual leaflets 2013 are assembled together to form the set of inner leaflets 2003. As shown, the commissures 2015 are attached together via sutures 2017, however, it should be understood that any appropriate means of attachment can be utilized, including (but not limited to) sutures, staples, and/or biocompatible adhesive. Each leaflet of the inner leaflet assembly 2003 is attached to the inner wall of the conduit 2005 via sutures 2019, however, it should be understood that any appropriate means of attachment can be utilized, including (but not limited to) sutures, staples, and/or biocompatible adhesive. A 4-hole bar 2021 is utilized as rigid/solid structure to reinforce the attachment between the inner leaflets 2003 and conduit 2005 wall. It is noted, however, that sutures alone or any other appropriate rigid/solid structure can be utilized to help reinforce this attachment. The inner leaflets 2003 can also incorporate reinforced tissue (not shown) such as layered, rolled, and/or folded tissue, as detailed in the description of FIGS. 14 and 15. Although three leaflets are shown assembled together in this particular example, any number of leaflets as described herein can be utilized.

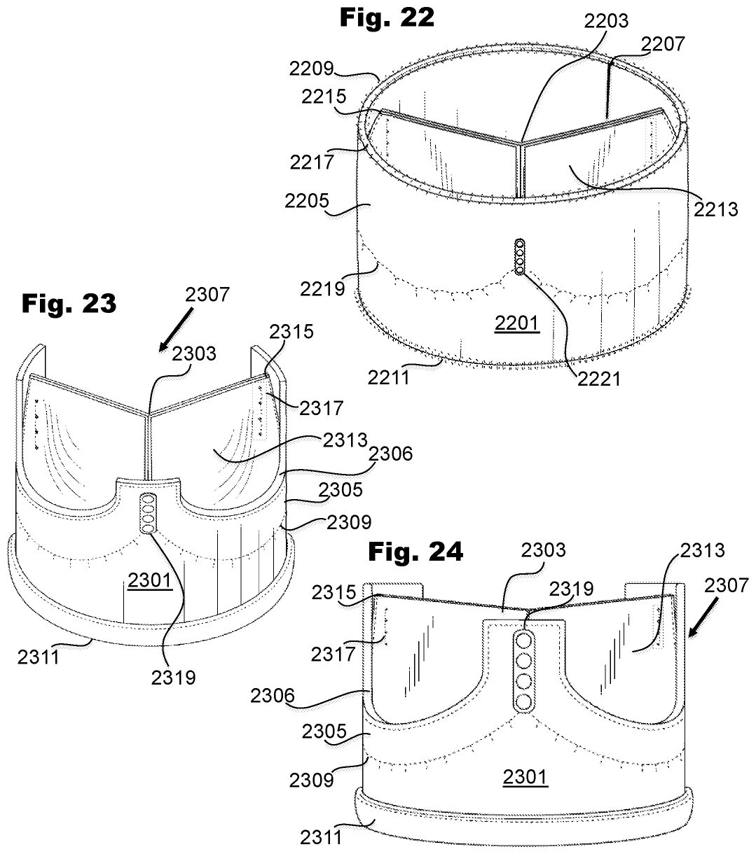

Provided in FIG. 22 is an example of a tissue-based valve 2201, having an inner leaflet assembly 2203 and a multi-layered conduit 2205. In this example, the conduit 2205 is two or more sheets of tissue with two ends adjoined together 2207 to form a tubular structure to house the inner leaflet assembly 2203. In addition, the plurality of sheets of tissue are attached together at the outflow edge 2209 and inflow edge 2221. Any appropriate means can be utilized to adjoin the edges of the sheets of tissue, including (but not limited to) sutures, staples, and/or biocompatible adhesive. The layered tissue of the conduit 2205 may include a biocompatible filler, as described herein.

The inner leaflet assembly 2203 of the tissue-based valve 2201 depicted in FIG. 22 can be constructed and attached in a similar manner to the inner leaflets of the embodiment portrayed in FIGS. 16 and 17. Three leaflets 2213 are assembled together to form the inner leaflet assembly 2203. As shown, the commissures 2215 are attached together via sutures 2217, however, it should be understood that any appropriate means of attachment can be utilized, including (but not limited to) sutures, staples, and/or biocompatible adhesive. The inner leaflet assembly 2203 is attached to the inner wall of the conduit 2205 via sutures 2219, however, it should be understood that any appropriate means of attachment can be utilized, including (but not limited to) sutures, staples, and/or biocompatible adhesive. A 4-hole bar 2221 is utilized as a rigid/solid structure to reinforce the attachment between the inner leaflet assembly 2203 and conduit 2205 wall. It is noted, however, that sutures alone or any other appropriate rigid/solid structure can be utilized to help reinforce this attachment. The inner leaflet assembly 2203 can also incorporate reinforced tissue (not shown) such as layered, rolled, and/or folded tissue, as detailed in the description of FIGS. 14 and 15. Although three leaflets are shown assembled together in this particular example, any number of leaflets as described herein can be utilized.

FIGS. 23 and 24 depict an embodiment of a tissue-based valve 2301 in perspective and front-elevation views, respectively. The tissue-based valve 2301 incorporates an inner leaflet assembly 2303 within a conduit 2305. In this example, the conduit 2305 is a sheet of tissue with two ends adjoined together to form a tubular structure to house the inner leaflet assembly 2303. Any appropriate means can be utilized to adjoin the two ends of the sheet of tissue, including (but not limited to) sutures, staples, and/or biocompatible adhesive. The outflow edge 2306 of conduit 2305 has is contoured such that there is recessed portion 2307 in between adjacent commissures 2315. As shown here, the outflow edge 2307 has a curved contour that runs along the sutures 2309 that provide attachment of the leaflet cusp edge to the conduit sidewall, although any shape of contour that provides a recess can be utilized. Further, the size of recessed portion 2307 can vary. Recessed portions of the conduit wall allow coronary access when the valve is implanted in the aortic position, but still maintain a suitable attachment between the set of inner leaflets 2303 and conduit 2305.

In this example, the conduit 2305 incorporates reinforced tissue along the and inflow edge 2311. In particular, these figures depict folded tissue, although it should be understood that reinforced tissue could be layered, folded, and/or rolled. Reinforced tissue can provide strength at the inflow edge 2311. In addition, the reinforced tissue can help strengthen the eventual connection between the tissue-based valve 2301) to the local tissue at the site of implantation (e.g., within the recipient's vasculature). Folded, rolled, and/or layered tissue may include a biocompatible filler, as described herein. Furthermore, reinforced tissue can be provided on the outflow edges above the commissures.

The inner leaflet assembly 2303 of the tissue-based valve 2301 depicted in FIGS. 23 and 24 can be constructed and attached in a similar manner to the inner leaflets of the embodiment portrayed in FIGS. 16 and 17. Three individual leaflets 2313 are assembled together to form the inner leaflet assembly 2303. As shown, the commissures 2315 are attached together via sutures 2317, however, it should be understood that any appropriate means of attachment can be utilized, including (but not limited to) sutures, staples, and/or biocompatible adhesive. The inner leaflet assembly 2303 is attached to the inner wall of the conduit 2305 via sutures 2309, however, it should be understood that any appropriate means of attachment can be utilized, including (but not limited to) sutures, staples, and/or biocompatible adhesive. A 4-hole bar 2319 is utilized as a rigid/solid structure to reinforce the attachment between the inner leaflets 2303 and conduit 2305 wall. It is noted, however, that sutures alone or any other appropriate rigid/solid structure can be utilized to help reinforce this attachment. The inner leaflets 2303 can also incorporate reinforced tissue (not shown) such as layered, rolled, and/or folded tissue, as detailed in the description of FIGS. 14 and 15. Although three leaflets are shown assembled together in this particular example, any number of leaflets as described herein can be utilized.

A tissue-based heart valve is to be inserted within animal vasculature to replace or assist a dysfunctional valve. In some instances, a tissue-based heart valve is to be inserted within an aortic root to replace a dysfunctional aortic valve, where the forces related to systole and diastole pressures are strong and repetitive. Because tissue-based heart valves are generally composed of soft tissue, these valves can lack sufficient rigidity to withstand strong pulsatile pressures in the aortic root and elsewhere. Thus, an implanted tissue-based heart valve without reinforcement can collapse, causing great damage and preventing the valve from properly integrating within the site of implantation. Accordingly, several devices are directed to providing a reinforcing tissue structure that provides structural rigidity capable of withstanding constricting and pulsatile forces associated with blood pressure. A reinforcing tissue structure can maintain a tissue-based heart valve's shape and functionality while under stress from the blood pressure forces.

Various reinforced tissue-based valves of the present disclosure have the rigidity and support to withstand pulsatile forces such that the heart valve can maintain a valvular shape. Accordingly, these reinforced tissue-based valves have enough compressive strength to prevent collapse. Likewise, these reinforced tissue-based valves have enough fatigue strength such that it is able to withstand pulsatile pressures associated with systole and diastole. As known in the art, pressures within aortic root can be approximately 120 systolic mmHg in a typical human, and can reach above 150 systolic mmHg or even 180 systolic mmHg in an individual suffering from severe hypertension. Accordingly, a reinforced tissue-based heart valve is able to withstand pressures of at least 100 mmHg, 110 mmHg, 120 mmHg, 130 mmHg, 140 mmHg, 150 mmHg, 160 mmHg, 170 mmHg, or 180 mmHg.

### Delivery and Expandability of Tissue-Based Valves

Described herein are a number of various methods of delivering a tissue-based valve to the site of deployment. A method can be performed on any suitable recipient, including (but not limited to) humans, other mammals (e.g., porcine), cadavers, or anthropomorphic phantoms, as would be understood in the art. Accordingly, methods of delivery include both methods of treatment (e.g., treatment of human subjects) and methods of training and/or practice (e.g., utilizing an anthropomorphic phantom that mimics human vasculature to perform method). Methods of delivery include (but not limited to) open heart surgery and transcatheter delivery.

When a transcatheter delivery system is used, any appropriate approach may be utilized to reach the site of deployment, including (but not limited to) a transfemoral, subclavian, transapical, or transaortic approach. A catheter containing a support ring and/or regenerative valve can be delivered via a guidewire to the site of deployment. At the site of deployment, a support ring and/or regenerative valve can be released from the catheter and then expanded into form. A number of expansion mechanisms can be utilized, such as (for example) an inflatable balloon, mechanical expansion, or utilization of a self-expanding device. Particular shape designs and radiopaque regions on the valves can be utilized to monitor the expansion and implementation.

Delivery and employment of a tissue-based valve may be utilized in a variety of applications. In some applications, a tissue-based valve is delivered to a site for valve replacement and/or supplement. Sites include the aortic valve, mitral valve, tricuspid valve, and pulmonary valve.

A tissue-based heart valve can be expandable such that the valve is to be surgically implanted via a transcatheter. Accordingly, a tissue-based heart valve is in a crimped or unexpanded form prior to implantation and expanded into functional form at the site of implantation.

Tissue portions of a valve can be crimped and/or folded into an unexpanded or crimped state such that it can fit within a transcatheter. When a valve incorporates a mesh frame, the mesh frame is elongated into an unexpanded form such that it can fit within a transcatheter. When a tissue-based valve reaches the site of implantation, a balloon or other means is used to expand a tissue-based valve, including the tissue portions and the mesh frame (if appropriate) into its functional form. When a tissue-based valve with a mesh frame reaches the site of implantation, a self-expanding mesh frame can be used to expand a tissue-based valve, including the tissue portions and the mesh frame into its functional form. Once expanded into its functional form, a tissue-based valve can be implanted into the appropriate space. When replacing an aortic valve, a tissue-based valve should be situated within the aortic root such that the base is located at the aortic annulus, the top of the leaflets are located at the sinotubular junction.

### Regenerative Tissue

Several tissue-based valves described herein can be formed of regenerative tissue. Regenerative tissue to be utilized in a tissue-based heart valve can be any appropriate formulation of regenerative tissue as understood in the art. In various situations, regenerative tissue is formulated in vitro. In some situations, regenerative tissue is autologous (i.e., generated from tissue and or cells of the recipient to be treated). In some situations, regenerative tissue is allogenic (i.e., generated from a source other than the individual to be treated). When allogenic tissue is be used, appropriate measures to mitigate immunoreactivity and/or rejection of the tissue may be necessary. Regenerative tissue may be decellularized animal tissue and/or extracellular matrix.

Regenerative tissue can be formulated such that a regenerative tissue-based heart valve is able to adapt and integrate within the site of implantation. In many situations, regenerative tissue provides a scaffold such that host tissue integrates and grows into a native-mimicking valve and conduit. In some situations, a regenerative tissue-based heart valve is formulated to resist thrombosis and pannus formation. In some situations, a regenerative tissue-based heart valve is “trained” in bioreactor systems that simulate physiological and mechanical pressures that occur in the vasculature, including the aortic root.

Regenerative tissue can also be formulated on a scaffold such that the host tissue integrates with the implant, which grows into native-mimicking heart valve and conduit. Scaffolds can be biodegradable such that when implanted and/or a short time after implantation, the scaffold degrades. A number of scaffold matrices can be used, as understood in the art. In some situations, a synthetic polymer is used, such as (for example) polyglycolic acid (PGA), polylactic acid (PLA), poly-D-lactide (PDLA), polyurethane (PU), poly-4-hydroxybutyrate (P4HB), and polycaprolactone (PCL). In some situations, a biological matrix is used, which can be formulated from a number of biomolecules including (but not limited to) collagen, fibrin, hyaluronic acid, alginate, and chitosan. It should be understood that various scaffold matrices can be combined and utilized.

A number of cell sources can be utilized in formulating regenerative tissue. Cell sources include (but are not limited to) mesenchymal stem cells (e.g., derived from bone marrow), cardiac progenitor cells, endothelial progenitor cells, adipose tissue, vascular tissues, amniotic fluid-derived cells, and cells differentiated from pluripotent stem cells (including embryonic stem cells). Vascular tissue can be derived from peripheral arteries and/or umbilical veins, which can be used to isolate endothelial cells and myofibroblasts for regenerative tissue formulation. In some situations, pluripotent stem cells are induced into a pluripotent state from a mature cell (e.g., fibroblasts). In several situations, cells are sourced from an individual to be treated, which reduces concerns associated with allogenic sources.

A regenerative tissue-based heart valve can be used to be inserted within the vasculature (e.g., at the aortic root) to replace or assist a dysfunctional valve, especially where the forces related to systole and diastole pressures are extremely strong and repetitive. Regenerative tissue-based heart valves are generally composed of soft tissue and are highly plastic and can lack sufficient rigidity to withstand strong pulsatile pressures in the aortic root and elsewhere. Thus, a newly implanted regenerative heart valve can collapse, causing great damage and preventing the valve from properly integrating at the site of implantation. Accordingly, a reinforced regenerative tissue-based valve that has structural rigidity capable of withstanding the constricting and pulsatile forces associated with blood pressure can be used. Reinforcing elements may be able to maintain a regenerative heart valve's shape and functionality while under stress from the blood pressure forces.

### Assembly of Tissue-Based Heart Valve with Reinforcing Tissue Structure

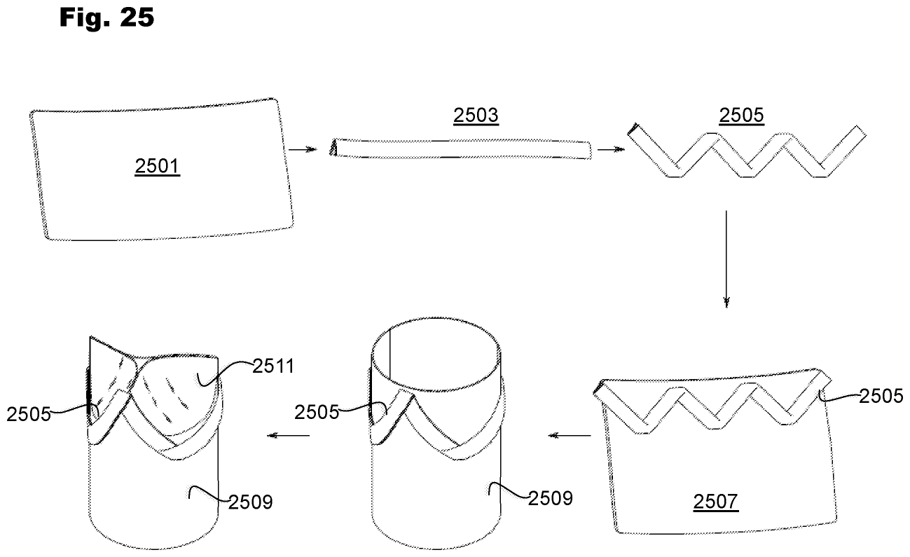

Provided in FIG. 25 is an example of a method to assemble a tissue-based valve having an attached reinforcing tissue structure. As shown in the figure, a first sheet of tissue 2501 is provided to serve as a source for a reinforcing tissue structure. Any appropriate animal tissue may be utilized, including (but not limited to) bovine pericardium, porcine pericardium, equine pericardium, or human tissue. Although a rectangular sheet of tissue is shown, any appropriate shape or size may be utilized in accordance with various embodiments.

As shown in the Figure, the first sheet of tissue 2501 is folded over several times to form a thickened tissue structure 2503. Other methods to thicken a tissue structure may be performed, such as (for example) layering several strips of tissue on top of one another, growing tissue into a thicker structure, and/or stuffing strips of tissue with a biocompatible filler. In some instances, a metallic mesh or other rigid structure may be inserted within or attached onto a thickened tissue structure, which may provide further structural support.

After forming an elongated and thickened tissue structure 2503, it can be formed into a zig-zag-like pattern 2505 and placed onto second sheet of tissue (2507) such that the zig-zag-like thickened tissue structure 2505 is situated towards one end. The second sheet of tissue 2507 can be rounded into a cylindrical tube to form the sidewalls 2509 of a tissue-based valve. The upper portions of the cylindrical tissue can be pinched and crimped such that a number of leaflets 2511 are formed with the zig-zag-like thickened tissue structure 2505 at the base of the leaflets. Sutures and/or a biocompatible adhesive may be utilized to hold the various components of the tissue-based valve together.

While specific examples of a method to assemble a tissue-based valve having an attached reinforcing tissue structure are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for assembling a tissue-based valve appropriate to the requirements of a given application can be utilized in accordance with various embodiments of the invention.

### Implantation of Tissue-Based Valve with Inner Leaflet Assembly

A tissue-based valve with inner leaflet assembly was prepared utilizing bovine pericardium. The tissue-based valve comprised an inner leaflet assembly having three leaflets, each leaflet with a free edge having length longer than the minimum length for coaptation. The leaflet assembly was constructed within a tissue-based conduit in which the cusp edge of each leaflet was sutured to the sidewall. The tissue-based valve further comprised three 4-hole bars, each sutured to the conduit sidewall and a commissure.

The tissue-based valve with inner leaflet assembly was implanted within sheep within the aorta, replacing the native aortic valve, where it is maintained for ten months until removed for examination. At ten months, the valve was fully functional and maintained intact, with little to no signs of damage. The sheep was healthy during the ten-month period. These results suggest that the tissue-based valve with inner leaflet assembly was functional and durable.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. An implantable heart valve device, comprising:
a conduit formed of animal tissue into a cylindrical shape having a side wall with an inner face and an outer face; and
an inner leaflet assembly formed of animal tissue comprising a plurality of leaflets, each leaflet having a cusp edge, a free edge, and a belly;
wherein a portion of the cusp edge of each leaflet is connected with a portion of a cusp edge of another leaflet to form a plurality of commissures;
wherein the cusp edge of each leaflet of the leaflet assembly is further connected with an inner face of the sidewall; and
wherein the free edges of leaflet assembly are capable of coapting together.

2. The device as in claim 1, wherein the inner leaflet assembly consists of 2, 3, 4, or 5 leaflets.

3. The device as in claim 1, wherein each leaflet of the inner leaflet assembly comprises a sheet of tissue having a free edge connected, a cusp edge, and a belly, the cusp edge contoured in a rounded line; wherein the free edge and the cusp edge are connected at two points forming two corners that are commissure meeting points, wherein each commissure meeting point is the connection location with at least one other leaflet to form a commissure; and wherein each commissure meeting point incorporates thickened tissue on an outflow face of the sheet of tissue.

4. The device as in claim 1, wherein the free edge of each leaflet has length that is between about 1.1× and 2× longer than the minimum distance for coaptation between the leaflet's commissures.

5. The device as in claim 1, wherein the cusp edge of at least one leaflet has a tissue sleeve attached thereupon.

6. The device as in claim 1, wherein at least one commissure of the plurality of commissures incorporates thickened tissue on an outflow face of at least one leaflet of the inner leaflet assembly.

7. The device as in claim 6, wherein the thickened issue is a side tab or a top tab that is extended from the commissure meeting points and is rolled or folded back onto the outflow face of the leaflet.

8. The device as in claim 6, wherein the thickened tissue is layers of tissue attached together and attached upon the outflow face of the leaflet at its commissure.

9. The device as in claim 6, wherein the thickened tissue incorporates a biocompatible filler.

10. The device as in claim 9, wherein the biocompatible filler comprises nitinol, cobalt-chromium, titanium, stainless steel, poly(lactic-co-glycolic) acid (PLGA), polyvinylchloride (PVC), polyethylene (PE), polypropylene (PP), polytetrafluoroethylene (PTFE), polyurethane (PU), polyethylene terephthalate (PET), polyether sulfone (PES), polyglycolic acid (PGA), polylactic acid (PLA), poly-D-lactide (PDLA), poly-4-hydroxybutyrate (P4HB), or polycaprolactone (PCL).

11. The device as in claim 6, wherein the thickened tissue has thickness of about 1.5×, 2×, 2.5×, 3×, 4×, or 5× the thickness of the leaflet.

12. The device as in claim 1, wherein the attachment between at least one commissure and the conduit wall is reinforced with a rigid structure.

13. The device as in claim 1, wherein the attachment between at least one commissure and the conduit wall is marked with a radiopaque structure.

14. The device as in claim 12, wherein the rigid structure or the radiopaque structure is a 4-hole bar.

15. The device as in claim 1, wherein the inner leaflet assembly is assembled within the conduit such that the free edges and bellies of each leaflet are mitigated from contacting an inner wall of the conduit when implanted and functioning.

16. The device as in claim 1, wherein the conduit is formed from a sheet of tissue connected at two opposite edges to form the cylindrical shape.

17. The device as in claim 1, wherein the conduit comprises a plurality of sheets of tissue layered and connected together to form a thickened conduit wall.

18. The device as in claim 17, wherein the thickened conduit wall, the thickened outflow edge, or the thickened inflow edge incorporates a biocompatible filler.

19. The device as in claim 1, wherein the conduit has an outflow edge that incorporates rolled, folded, or layered tissue to form thickened outflow edge.

20. The device as in claim 1, wherein the conduit has an inflow edge that incorporates rolled, folded, or layered tissue to form thickened inflow edge.

21. The device as in claim 1, wherein the conduit has an outflow edge that is contoured with at least one recessed portion in between a pair of adjacent commissures.

22. A leaflet for use within an implantable heart valve device, comprising:
a sheet of tissue having a free edge connected, a cusp edge, and a belly, the cusp edge contoured in a rounded line;
wherein the free edge and the cusp edge are connected at two points forming two corners that are commissure meeting points, wherein each commissure meeting point is for attachment with at least one other leaflet to form a commissure; and
wherein each commissure meeting point incorporates thickened tissue on an outflow face of the sheet of tissue.

23. The leaflet as in claim 22, wherein the cusp edge of at least one leaflet has a tissue sleeve attached thereupon.

24. The leaflet as in claim 22, wherein the thickened issue is a side tab or a top tab that is extended from the commissure meeting points and is rolled or folded back onto the outflow face of the leaflet.

25. The leaflet as in claim 22, wherein the thickened tissue is layers of tissue attached together and attached upon the outflow face of the leaflet at its commissure.

26. The leaflet as in claim 22, wherein the thickened tissue incorporates a biocompatible filler.

27. The leaflet as in claim 26, wherein the biocompatible filler comprises nitinol, cobalt-chromium, titanium, stainless steel, poly(lactic-co-glycolic) acid (PLGA), polyvinylchloride (PVC), polyethylene (PE), polypropylene (PP), polytetrafluoroethylene (PTFE), polyurethane (PU), polyethylene terephthalate (PET), polyether sulfone (PES), polyglycolic acid (PGA), polylactic acid (PLA), poly-D-lactide (PDLA), poly-4-hydroxybutyrate (P4HB), or polycaprolactone (PCL).

28. The leaflet as in claim 22, wherein the thickened tissue has thickness of about 1.5×, 2×, 2.5×, 3×, 4×, or 5× the thickness of the leaflet.

29. A leaflet assembly comprising a plurality of leaflets as in claim 22, wherein a portion of the cusp edge of each leaflet is connected with a portion of a cusp edge of another leaflet to form a plurality of commissures.

30. The leaflet assembly as in claim 29, wherein the plurality of leaflets consists of 2, 3, 4, or 5 leaflets.

31. The leaflet assembly as in claim 29, wherein the free edge of each leaflet has length that is between 1.1× and 2× longer than the minimum distance for coaptation between the leaflet's commissures.
